(19) United States
(12) Patent Application Publication
(10) Pub. No.: US 2024/0374536 A1
(43) Pub. Date: Nov. 14, 2024

(54) LOW-CARBON, GREEN AND EFFICIENT PREPARATION METHOD OF SOYBEAN OIL BODY-CURCUMIN EMULSION

(71) Applicant: Beijing Technology and Business University, Beijing (CN)

(72) Inventors: Duoxia Xu, Beijing (CN); Yanbo Wang, Beijing (CN); Baoguo Sun, Beijing (CN); Wenwen Lv, Beijing (CN); Wei Chen, Beijing (CN); Xiaoyu Li, Beijing (CN); Shaojia Wang, Beijing (CN); Bei Wang, Beijing (CN)

(21) Appl. No.: 18/657,837

(22) Filed: May 8, 2024

(30) Foreign Application Priority Data
May 9, 2023 (CN) 2023105140549

(51) Int. Cl. A61K 31/12; A61K 9/107; A61K 47/44

(52) U.S. Cl. CPC A61K 31/12; A61K 9/107; A61K 47/44

(57) ABSTRACT

A low-carbon, green and efficient preparation method for soybean oil body-curcumin emulsion includes following steps: soybeans are mixed with NaHCO₃ solution at a ratio of 1:7, soaked in the NaHCO₃ solution at 4° C. for 20 h and blended with a blender for 6 min to obtain soybean slurry, pH of the soybean slurry is adjusted to 11.0, then the soybean slurry is stirred for 2 h at 50° C. in a water bath to obtain stirred slurry, the stirred slurry is filtered to obtain a suspension, and the suspension is centrifuged to obtain a soybean oil body; curcumin is dissolved with 0.2 M NaOH and then mixed with the soybean oil body to obtain a mixed emulsion, and pH of the mixed emulsion is adjusted to 6.5-8.0 and then the mixed emulsion is stirred for 30 min in dark to obtain the soybean oil body-curcumin emulsion.

### TECHNICAL FIELD

The disclosure relates to the field of food industry, and more particularly to a low-carbon, green and efficient preparation method of soybean oil body-curcumin emulsion.

### BACKGROUND

Curcumin is a low-molecular-weight lipophilic polyphenolic compound in rhizomes of turmeric (Curcuma longa L). The turmeric belongs to zingiberaceae, and is used as a spice, a flavoring agent, a coloring agent, a preservative, and a traditional medicine in Southeast Asia and other regions. The curcumin has a variety of biologically active effects, including antioxidant, anti-inflammatory, anti-tumor, and anti-atherosclerosis, etc. The curcumin has received widespread attention in the field of food development and pharmacological research, and has been allowed to be used as a natural pigment and a functional ingredient in functional foods by the European Union, China and other countries and relevant organizations. However, the curcumin has a low solubility in water, and will decompose and discolor when exposed to light, heat and oxygen, and its bioavailability is low, which greatly limit its application in the food industry. Design of corresponding carriers and construction of carrier systems to improve stability and bioavailability of the curcumin have become research hotspots among domestic and foreign scholars and the field of food science in recent years, in view of problems of the low solubility, poor stability and a fast metabolism of the curcumin.

Soybean, as a major oilseed crop, contains a large number of oil bodies within its seed, the oil bodies contain about 15% saturated fatty acids and 80% unsaturated fatty acids. A soybean oil body contains triglyceride inside, and is covered by a biofilm composed of phospholipids and proteins outside. The oil bodies can be evenly dispersed in water, forming a natural oil-in-water (O/W) emulsion with good stability, due to natural monolayer of phospholipids and proteins. In recent years, the soybean oil body has received more and more attention. The soybean oil body is rich in functional components such as polyunsaturated fatty acids, phospholipids, tocopherols and phytosterols, which has great application potential in food. Meanwhile, a hydrophobic core of the soybean oil body can be used to dissolve non-polar biological active substances such as oil-soluble vitamins and polyphenols, offering a possibility of the soybean oil body as a natural carrier. However, this characteristic has not been fully utilized due to a closed structure of the soybean oil body, and how to load the curcumin and other active substances into the soybean oil body on a basis of maintaining the natural structure and stability of the soybean oil body has become a key to its application.

### SUMMARY

The disclosure aims at providing a low-carbon, green and efficient preparation method of soybean oil body-curcumin emulsion, which uses soybean oil bodies to prepare emulsion, raw materials are green and natural, and a process of emulsion preparation is simple with less energy consumption, curcumin encapsulation rate is high, and emulsion stability is good.

The disclosure is realized by technical solutions as follows.

The low-carbon, green and efficient preparation method of the soybean oil body-curcumin emulsion includes following steps:
S1, extraction of a soybean oil body: soybeans are mixed with 0.1 mole per liter (M) sodium bicarbonate (NaHCO₃) solution, the soybeans are soaked in the NaHCO₃ solution at 4 degrees Celsius (° C.) for 20 hours (h), and then are stirred and chopped for 6 minutes (min) by a blender to obtain soybean slurry, pH of the soybean slurry is adjusted and then the soybean slurry is stirred in a water bath to obtain stirred soybean slurry, the stirred soybean slurry is filtered to obtain an oil body suspension, and the soybean oil body is obtained by centrifugating the oil body suspension for 20 min; and
S2, preparation of the soybean oil body-curcumin emulsion: curcumin is dissolved with sodium hydroxide (NaOH) solution to obtain curcumin solution, the curcumin solution is mixed with the soybean oil body to obtain a mixed emulsion, pH of the mixed emulsion is adjusted and then the mixed emulsion is stirred in dark for 30 min to obtain the soybean oil body-curcumin emulsion.

In an embodiment, the soybeans are mixed with the 0.1 M NaHCO₃ solution in a weight ratio of 1:7 in the step S1. A system viscosity is determined by a ratio of raw materials to extraction media. When a system has a low water content and a high viscosity (i.e., the ratio is less than 1:7), it will lead to aggregation of soybean oil bodies and a lower extraction rate; when the system viscosity is too low (i.e., the ratio is greater than 1:7), it will result in a waste of water resources and an increase in production costs.

In an embodiment, the pH of the soybean slurry is 11.0 in the step S1. The soybean oil body with high purity can be obtained at a high alkaline pH, however, when the pH of the soybean slurry is higher than 11.0, surface proteins of the soybean oil body may be removed, and when the pH of the soybean slurry is close to neutral, the soybean oil body contains a large number of proteins and has a low purity.

In an embodiment, in the step S1, the soybean slurry is stirred in the water bath at 50° C. for 2 h, it is beneficial to fully exposing the soybean oil body to water, and allowing water molecules to penetrate into cellular networks for more efficient extraction.

In an embodiment, in the step S1, the oil body suspension is centrifugated at 4° C. with a rotational speed of 10000 revolutions per minute (rpm). Centrifugation at a low temperature is conducive to maintaining structure of the soybean oil body, a low centrifugation speed will cause emulsification of small-sized soybean oil bodies, and a high centrifugation speed is prone to cause the aggregation of the soybean oil bodies and destruction of natural structure.

In an embodiment, in the step S2, the curcumin is dissolved with the NaOH solution at a concentration of 0.2 M. High concentration NaOH solution has high alkalinity and may destroy the structure of the soybean oil body when mixed with the soybean oil body, while low concentration NaOH solution has low alkalinity and poor solubility of the curcumin.

In an embodiment, in the step S2, a weight ratio of the curcumin to the soybean oil body is 5:1 to 1:5. When a proportion of the soybean oil body is too low in a system (i.e., the weight ratio is less than 1:1), the soybean oil body cannot completely encapsulate the curcumin and the emulsion stability is poor; when a proportion of the soybean oil body in a system is too high (i.e., the weight ratio is greater than 1:5), the encapsulation capacity of the curcumin is low, which is not conducive to applications of the soybean oil body-curcumin emulsion.

In an embodiment, in the step S2, the pH of the mixed emulsion is adjusted to 6.5-8.0. When the pH of the mixed emulsion is low (i.e., less than 6.5), it is close to an isoelectric point of oil body proteins, the proteins are aggregated, and emulsion viscosity become larger; when the pH of the mixed emulsion is high (i.e., greater than 8.0), the curcumin has better solubility, and is not fully loaded into the soybean oil body.

### Beneficial Effect

The disclosure provides the low-carbon, green and efficient preparation method of the soybean oil body-curcumin emulsion. The soybean oil body is extracted with 0.1 M NaHCO₃ as a medium, and the pH of the soybean slurry is adjusted to 11.0 to obtain the soybean oil body with complete structure and high purity. Curcumin emulsion is prepared from the green and natural soybean oil body, and the soybean oil body-curcumin emulsion with good stability and high encapsulation rates can be obtained by adjusting the pH of the mixed emulsion. The preparation process is low-carbon, green and simple, and the pH value of the system is neutral or weakly alkaline. The low-carbon, green and efficient preparation method of the soybean oil body-curcumin emulsion is developed and suitable for food industry.

### BRIEF DESCRIPTION OF DRAWINGS

FIG. 1A to FIG. 1C illustrate physical stability of soybean oil body-curcumin emulsion prepared in an embodiment 1.

FIG. 2A to FIG. 2E illustrate physical stability of soybean oil body-curcumin emulsion prepared in an embodiment 2.

FIG. 3A to FIG. 3D illustrate physical stability of soybean oil body-curcumin emulsion prepared in an embodiment 3.

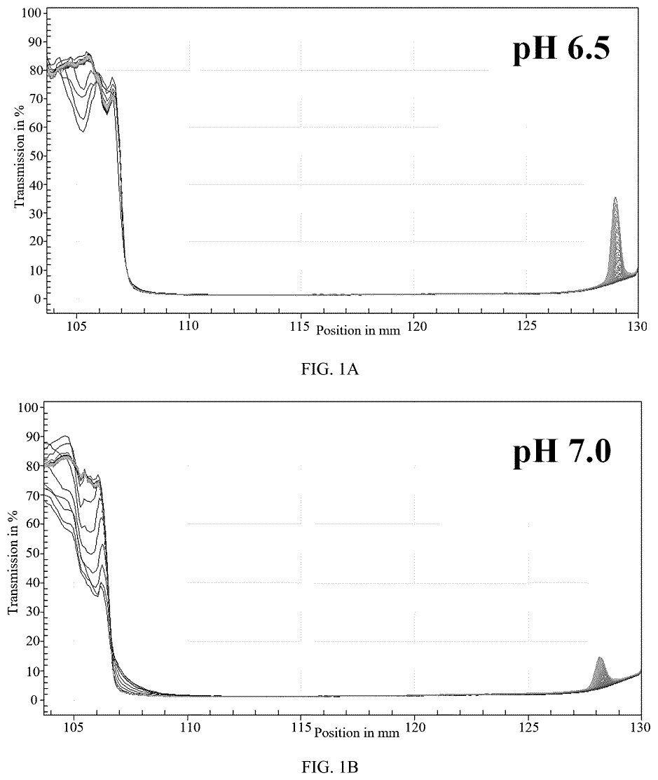

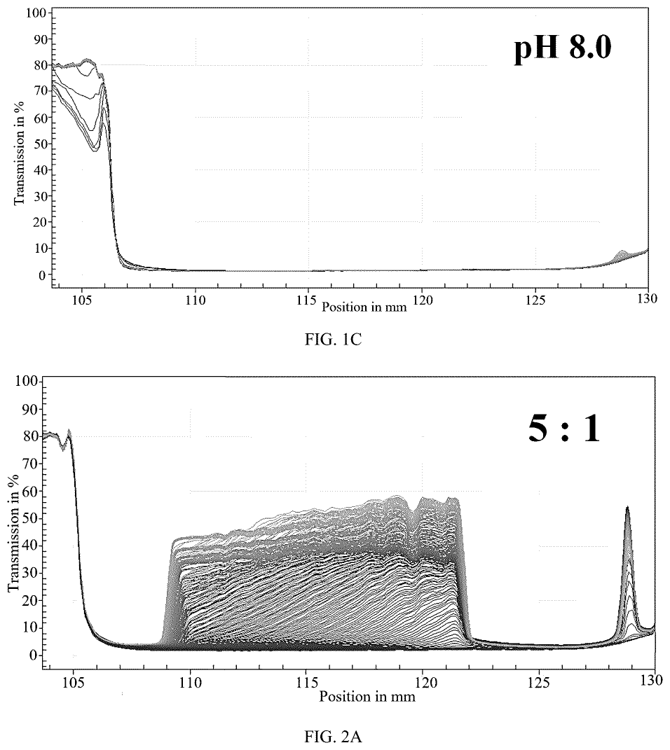

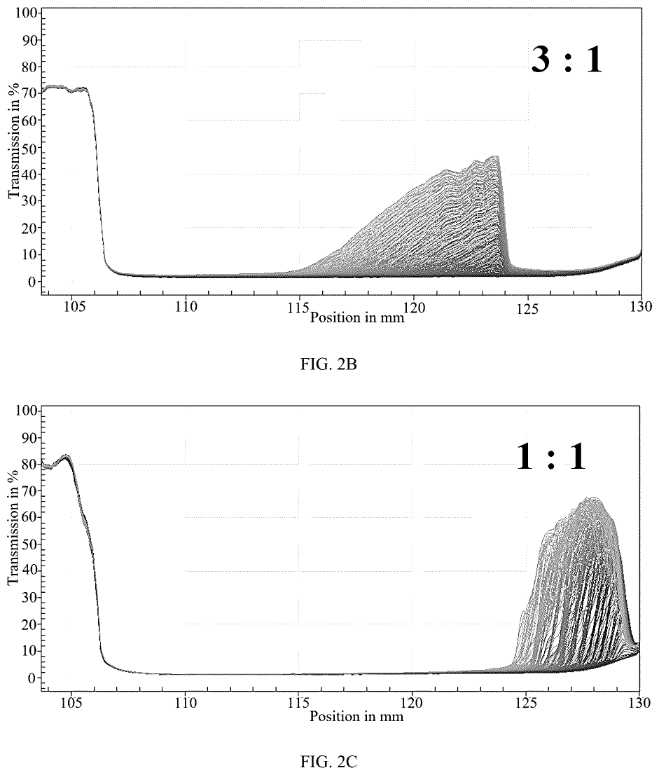

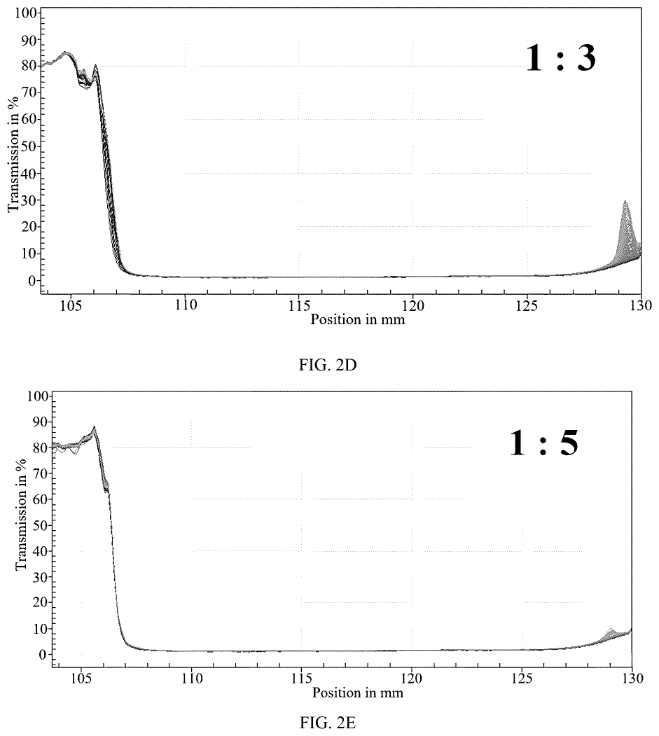

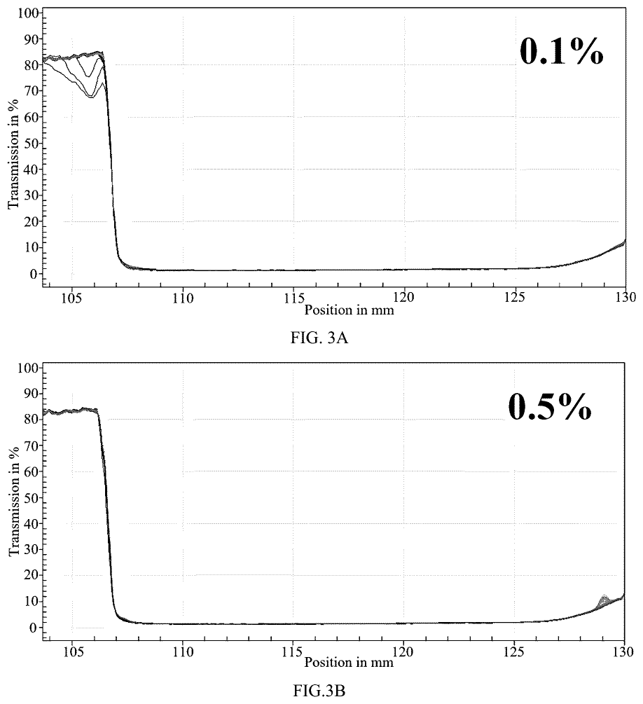

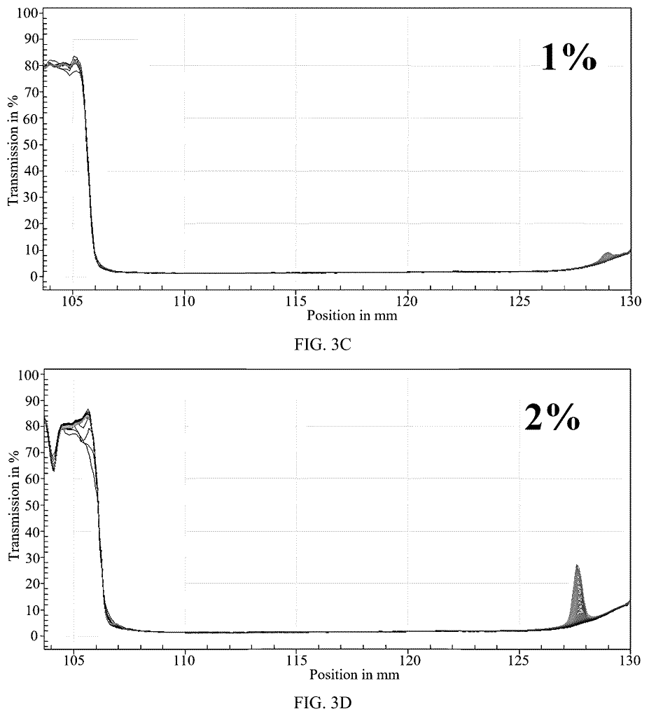

### DETAILED DESCRIPTION OF EMBODIMENTS

A detailed description of embodiments of the disclosure is below. The embodiments are implemented based on a technical solution of the disclosure, and detailed implementation methods and specific operation processes are provided, however, protection scope of the disclosure is not limited to the following embodiments.

### Embodiment 1

The soybean oil body-curcumin emulsion is prepared by the following steps.
(1) Extraction of a soybean oil body: soybeans are mixed with 0.1 M NaHCO₃ solution at a weight ratio of 1:7, the soybeans are soaked in the NaHCO₃ solution at 4° C. for 20 h, and then stirred and chopped for 6 min by a blender to obtain soybean slurry, pH of the soybean slurry is adjusted to 11.0 and then the soybean slurry is stirred in a water bath at 50° C. for 2 h to obtain stirred soybean slurry, the stirred soybean slurry is filtered to obtain an oil body suspension, and the oil body suspension is centrifugated at 4° C. for 20 min with a rotational speed of 10000 rpm to obtain the soybean oil body.
(2) Preparation of soybean oil body-curcumin emulsion: curcumin is dissolved with 0.2 M NaOH solution to obtain curcumin solution, the curcumin solution is mixed with the soybean oil body at a weight ratio of 1:5 to make a final concentration of the curcumin be 1%, thereby to obtain a mixed emulsion, pH of the mixed emulsion is separately adjusted to 6.5, 7.0 and 8.0, and then each mixed emulsion is stirred in dark for 30 min to obtain the soybean oil body-curcumin emulsion.

### Embodiment 2

The soybean oil body-curcumin emulsion is prepared by the following steps.

(1) Extraction of a soybean oil body: soybeans are mixed with 0.1 M sodium NaHCO₃ solution at a weight ratio of 1:7, the soybeans are soaked in the NaHCO₃ solution at 4° C. for 20 h and then are stirred and chopped for 6 min by a blender to obtain soybean slurry, pH of the soybean slurry is adjusted to 11.0 and then the soybean slurry is stirred in a water bath at 50° C. for 2 h to obtain stirred soybean slurry, the stirred soybean slurry is filtered to obtain an oil body suspension, and the oil body suspension is centrifugated at 4° C. for 20 min with a rotational speed of 10000 rpm to obtain the soybean oil body.
(2) Preparation of the soybean oil body-curcumin emulsion: curcumin is dissolved with 0.2 M NaOH solution to obtain curcumin solution, the curcumin solution is mixed with the soybean oil body at a weight ratio of 5:1, 3:1, 1:1, 1:3, and 1:5 separately to obtain mixed emulsions, pH of each mixed emulsion is adjusted to 7.0 and then each mixed emulsion is stirred in dark for 30 min to obtain the soybean oil body-curcumin emulsion.

### Embodiment 3

The soybean oil body-curcumin emulsion is prepared by the following steps.
(1) Extraction of a soybean oil body: soybeans are mixed with 0.1 M NaHCO₃ solution at a weight ratio of 1:7, the soybeans are soaked in the NaHCO₃ solution at 4° C. for 20 h and then are stirred and chopped for 6 min by a blender to obtain soybean slurry, pH of the soybean slurry is adjusted to 11.0 and then the soybean slurry is stirred in a water bath at 50° C. for 2 h to obtain stirred soybean slurry, the stirred soybean slurry is filtered to obtain an oil body suspension, and the oil body suspension is centrifugated at 4° C. for 20 min with a rotational speed of 10000 rpm to obtain the soybean oil body.
(2) Preparation of the soybean oil body-curcumin emulsion: curcumin is dissolved with 0.2 M NaOH solution to obtain curcumin solution, the curcumin solution is mixed with the soybean oil body at a weight ratio of 1:5 to make sure final concentrations of the curcumin are separately 0.1%, 0.5%, 1% and 2%, thereby to obtain mixed emulsions, pH of each mixed emulsion is adjusted to 7.0 and then each mixed emulsion is stirred in dark for 30 min to obtain the soybean oil body-curcumin emulsion.

### Physical and Chemical Properties and Stability Testing

1. A testing method for particle sizes: the particle size of the soybean oil body-curcumin emulsion obtained in each embodiment is gaged by a MICROTRAC S3500 laser diffraction analyzer. Wet measurement utilizes a principle of static light scattering, a sample is transported to a measurement unit by water with a refractive index 1.33 as a medium. The measurement is repeated 3 times to obtain an average value.
2. A testing method for zeta-potential: the zeta-potential of the soybean oil body-curcumin emulsion in each embodiment is gaged by a NANO-ZS90 laser particle size analyzer. To avoid multiple scattering effect, the soybean oil body-curcumin emulsion is diluted with phosphate buffer solution at corresponding pH to about 0.005% oil concentration, and refractive indexes of continuous phase and dispersed phase are set as 1.33 and 1.45 correspondingly.
3. A testing method for instability index: physical stability of the soybean oil body-curcumin emulsion in each embodiment is gaged by LUMiSizer® (it is a centrifugation-based tool that measures the size and stability of nano- and micron-sized dispersions) to accelerate instability. Operating conditions are temperature 25° C., injection volume 0.4 milliliters (mL), rotational speed 2500 rpm, total time 255 min, and time interval 30 seconds.
4. A testing method for curcumin encapsulation rate: the soybean oil body-curcumin emulsion in each embodiment is diluted 10 times with absolute ethanol to obtain a diluted emulsion, the diluted emulsion is centrifuged at 3000 rpm for 10 min to obtain a supernatant, an absorbance value of the supernatant is gaged at 425 nanometers (nm) wavelength through an ultraviolet spectrophotometer, and the amount of unencapsulated curcumin is calculated through standard curves.

$$Curcumin encapsulation rate = 100 \% \times ( initial amount of the curcumin - amount of the unencapsulated curcumin ) \text{} / initial amount of the curcumin .$$

TABLE 1
the curcumin encapsulation rate and the physical and chemical
properties of the soybean oil body-curcumin emulsion prepared
in the embodiment 1, embodiment 2 and embodiment 3.

| | Practical size | Zeta-Potential | Curcumin |
|---|---|---|---|
| Method | (nm) | (mV) | encapsulation rate |
| Embodiment 1 (pH = 6.5) | 420.23 ± 19.59 | −32.27 ± 0.53 | 88.53 ± 0.001 |
| Embodiment 1 (pH = 7.0) | 388.07 ± 14.04 | −36.30 ± 0.37 | 89.50 ± 0.003 |
| Embodiment 1 (pH = 8.0) | 362.07 ± 14.40 | −38.87 ± 0.25 | 88.56 ± 0.002 |
| Embodiment 2 (ratio of curcumin | 1564.00 ± 247.80 | −29.47 ± 1.76 | 86.87 ± 0.003 |
| to soybean oil body is 5:1) | | | |
| Embodiment 2 (ratio of curcumin | 476.8 ± 21.61 | −32.27 ± 0.24 | 87.03 ± 0.006 |
| to soybean oil body is 3:1) | | | |
| Embodiment 2 (ratio of curcumin | 437.67 ± 26.34 | −30.33 ± 0.73 | 87.01 ± 0.005 |
| to soybean oil body is 1:1) | | | |
| Embodiment 2 (ratio of curcumin | 396.00 ± 10.01 | −36.17 ± 0.40 | 87.77 ± 0.006 |
| to soybean oil body is 1:3) | | | |
| Embodiment 2 (ratio of curcumin | 388.07 ± 14.04 | −36.30 ± 0.37 | 89.50 ± 0.003 |
| to soybean oil body is 1:5) | | | |
| Embodiment 3 (curcumin | 384.40 ± 8.40 | −34.87 ± 0.21 | 83.72 ± 0.018 |
| concentration is 0.1%) | | | |
| Embodiment 3 (curcumin | 418.33 ± 17.05 | −35.90 ± 0.22 | 89.97 ± 0.003 |
| concentration is 0.5%) | | | |
| Embodiment 3 (curcumin | 388.07 ± 14.04 | −36.30 ± 0.37 | 89.50 ± 0.003 |
| concentration is 1%) | | | |
| Embodiment 3 (curcumin | 429.83 ± 7.31 | −36.20 ± 0.57 | 93.48 ± 0.003 |
| concentration is 2%) | | | |

It should be noted that the physical stability of the soybean oil body-curcumin emulsion is good when the pH in the step S2 is adjusted to 6.5, 7.0 and 8.0, when the pH is 8.0, the soybean oil body-curcumin emulsion has the smallest practical size, the largest absolute value of potential, and the highest encapsulation rate of 88.56%. When the ratio of the curcumin to the soybean oil body is 1:5, the soybean oil body-curcumin emulsion has the best stability, the smallest practical size and the largest absolute value of potential. When the curcumin concentration is 2%, the soybean oil body-curcumin emulsion shows good stability, practical size, potential and encapsulation rate. The soybean oil body-curcumin emulsion prepared by this method has good stability and high encapsulation rate. The disclosure successfully realizes the low-carbon, green and efficient preparation method for the soybean oil body-curcumin emulsion.

Apparently, those skilled in the art can make various modifications and variations to the disclosure without departing from the spirit and scope of the disclosure. Thus, when the modifications and the variations of the disclosure fall within the scope of the claims of the disclosure and technical equivalents thereof, the disclosure is intended to include such modifications and variations.

What is claimed is:

1. A preparation method of soybean oil body-curcumin emulsion, comprising following steps:
S1, extracting a soybean oil body: mixing soybeans with 0.1 mole per liter (M) sodium bicarbonate (NaHCO₃) solution, soaking the soybeans in the NaHCO₃ solution at 4 degrees Celsius (° C.) for 20 hours (h), stirring and chopping the soybeans soaked in the NaHCO₃ solution for 6 minutes (min) by a blender to obtain soybean slurry, adjusting pH of the soybean slurry and then stirring the soybean slurry in a water bath at 50° C. for 2 h to obtain stirred soybean slurry, filtering the stirred soybean slurry to obtain an oil body suspension, obtaining the soybean oil body by centrifugating the oil body suspension at 4° C. for 20 min with a rotational speed of 10000 revolutions per minute (rpm); and
S2, preparing the soybean oil body-curcumin emulsion: dissolving curcumin with sodium hydroxide (NaOH) solution to obtain curcumin solution, mixing the curcumin solution with the soybean oil body to obtain a mixed emulsion, adjusting pH of the mixed emulsion and then stirring the mixed emulsion in dark for 30 min to obtain the soybean oil body-curcumin emulsion.

2. The preparation method of the soybean oil body-curcumin emulsion as claimed in claim 1, wherein the soybeans are mixed with the 0.1 M NaHCO₃ solution in a weight ratio of 1:7 in the step S1.

3. The preparation method of the soybean oil body-curcumin emulsion as claimed in claim 1, wherein the pH of the soybean slurry is 11.0 in the step S1.

4. The preparation method of the soybean oil body-curcumin emulsion as claimed in claim 1, wherein the curcumin is dissolved with the NaOH solution at a concentration of 0.2 M in the step S2.

5. The preparation method of the soybean oil body-curcumin emulsion as claimed in claim 1, wherein a weight ratio of the curcumin to the soybean oil body is 5:1 to 1:5 in the step S2.

6. The preparation method of the soybean oil body-curcumin emulsion as claimed in claim 1, wherein the pH of the mixed emulsion is adjusted to 6.5 to 8.0 in the step S2.
